(19) United States
(12) Patent Application Publication
(10) Pub. No.: US 2005/0261596 A1
(43) Pub. Date: Nov. 24, 2005

(54) Passive switched capacitor high-pass filter for implantable cardiac device

(76) Inventor: Brian A. Smith, Apple Valley, MN (US)

Correspondence Address: SCHWEGMAN, LUNDBERG, WOESSNER & KLUTH, P.A., P.O. BOX 2938, MINNEAPOLIS, MN, 55402-0938, US

(21) Appl. No.: 10/852,655

(22) Filed: May 24, 2004

(51) Int. Cl. A61B 5/0402; A61N 1/36

(52) U.S. Cl. 600/510; 607/9; 607/5

(57) ABSTRACT

A sensing circuit for an implantable medical device is disclosed which enables a high-pass filtering circuit to be implemented as part of an integrated circuit chip. The high-pass filtering circuit utilizes a switched capacitor circuit so that the necessary circuit elements may have values within ranges suitable for chip fabrication.

### FIELD OF THE INVENTION

This invention pertains to cardiac rhythm management devices such as pacemakers and implantable cardioverter/defibrillators.

### BACKGROUND

Cardiac rhythm management devices are implantable devices that provide electrical stimulation to selected chambers of the heart in order to treat disorders of cardiac rhythm. A pacemaker, for example, is a cardiac rhythm management device that paces the heart with timed pacing pulses. The most common condition for which pacemakers are used is in the treatment of bradycardia, where the ventricular rate is too slow. Atrio-ventricular conduction defects (i.e., AV block) that are permanent or intermittent and sick sinus syndrome represent the most common causes of bradycardia for which permanent pacing may be indicated. If functioning properly, the pacemaker makes up for the heart's inability to pace itself at an appropriate rhythm in order to meet metabolic demand by enforcing a minimum heart rate and/or artificially restoring AV conduction. Other cardiac rhythm management devices are designed to detect atrial and/or ventricular tachyarrhythmias and deliver electrical stimulation in order to terminate the tachyarrhythmia in the form of a cardioversion/defibrillation shock or anti-tachycardia pacing. Certain combination devices may incorporate all of the above functionalities.

Cardiac rhythm management devices such as described above monitor the electrical activity of heart via one or more sensing channels so that pacing pulses or defibrillation shocks can be delivered appropriately. Such sensing channels include implanted leads which have electrodes disposed internally near the heart chamber to be sensed, which leads may also be used for delivering pacing pulses or defibrillation shocks. These sensing channels are designed to pick up biopotential signals and to detect the tissue depolarization that occurs when an atrium or ventricle contracts, which detection is referred to as an atrial or ventricular sense, respectively. The biopotential signal produced by cardiac activity, referred to as an electrogram signal, reflects the time course of both cardiac depolarization and repolarization as the heart beats. One way in which depolarization and repolarization waveforms can be distinguished is by their differing frequency contents. The usual practice is therefore to filter the electrogram signal before further processing in order to detect the depolarization signal. It is with methods and apparatus for such filtering that the present invention is concerned.

### BRIEF DESCRIPTION OF THE DRAWINGS

FIG. 1 is a block diagram of an exemplary cardiac rhythm management device for practicing the present invention.

FIG. 2 shows an example of a low-pass and high-pass filtering circuit.

FIG. 3 shows an example design scheme for a sensing channel.

FIG. 4 shows an improved design scheme for a sensing channel.

FIG. 5 illustrates a passive high-pass filter utilizing a switched capacitor.

FIG. 6 illustrates an alternate implementation of a switched capacitor passive high-pass filter.

### DETAILED DESCRIPTION

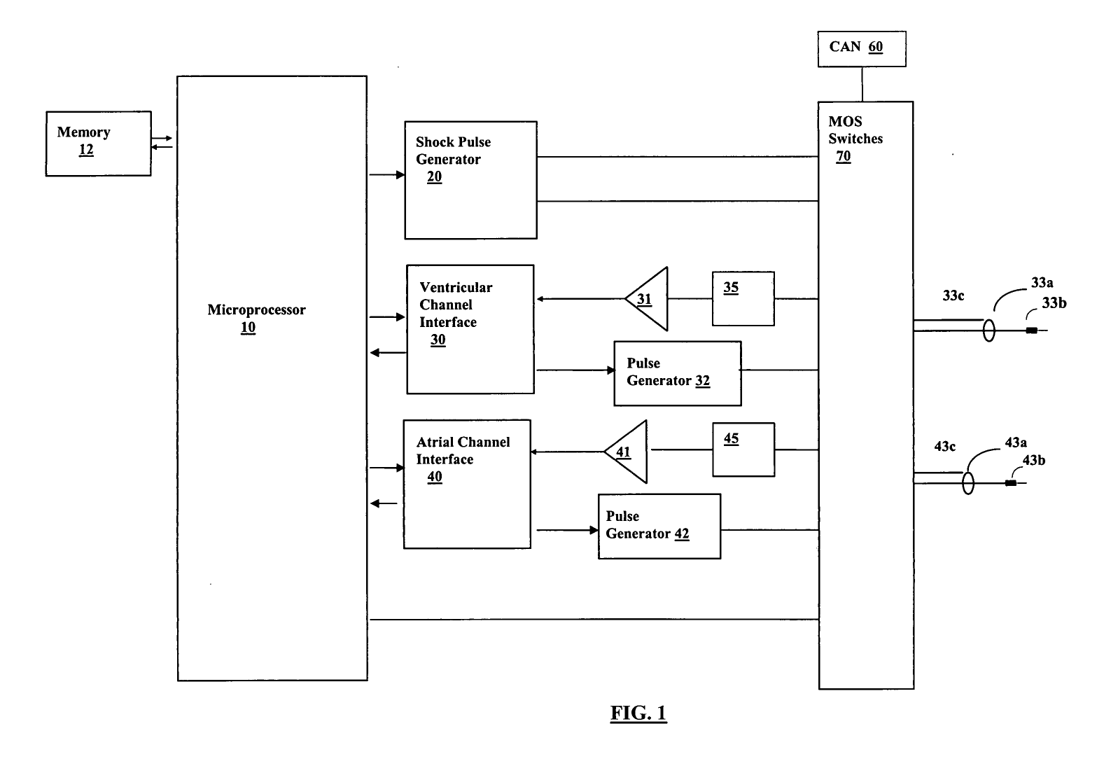

A block diagram of an implantable cardiac rhythm management device is shown in FIG. 1. Cardiac rhythm management devices are implantable devices that provide electrical stimulation to selected chambers of the heart in order to treat disorders of cardiac rhythm. Such devices are usually implanted subcutaneously on the patient's chest and connected to electrodes by leads threaded through the vessels of the upper venous system into the heart. An electrode can be incorporated into a sensing channel that generates an electrogram signal representing cardiac electrical activity at the electrode site and/or incorporated into a pacing or shocking channel for delivering pacing or shock pulses to the site.

The controller of the device is made up of a microprocessor 10 communicating with a memory 12 via a bidirectional data bus, where the memory 12 typically comprises a ROM (read-only memory) for program storage and a RAM (random-access memory) for data storage. The controller is capable of operating the device so as to deliver a number of different therapies in response to detected cardiac activity. The embodiment shown in FIG. 1 has two sensing/pacing channels, where a pacing channel is made up of a pulse generator connected to an electrode while a sensing channel is made up of the sense amplifier connected to an electrode. A MOS switch matrix 70 controlled by the microprocessor is used to switch the electrodes from the input of a sense amplifier to the output of a pulse generator. The switch matrix 70 also allows the sensing and pacing channels to be configured by the controller with different combinations of the available electrodes. The channels may be configured as either atrial or ventricular channels. In an example configuration, an atrial sensing/pacing channel includes ring electrode 43a and tip electrode 43b of bipolar lead 43c, filtering circuit 45, sense amplifier 41, pulse generator 42, and a channel interface 40. A ventricular sensing/pacing channel includes ring electrode 33a and tip electrode 33b of bipolar lead 33c, filtering circuit 35 sense amplifier 31, pulse generator 32, and a channel interface 30. The channel interfaces communicate bi-directionally with a port of microprocessor 10 and may include analog-to-digital converters for digitizing sensing signal inputs from the sensing amplifiers, registers that can be written to for adjusting the gain and threshold values of the sensing amplifiers, and registers for controlling the output of pacing pulses and/or changing the pacing pulse amplitude. A shock pulse generator 20 is also interfaced to the controller for delivering defibrillation shocks through electrodes selected by the switch matrix. In the illustrated embodiment, the device is equipped with bipolar leads that include two electrodes which are used for outputting a pacing pulse and/or sensing intrinsic activity. Other embodiments may employ unipolar leads with single electrodes for sensing and pacing. The switch matrix 70 may configure a channel for unipolar sensing or pacing by referencing an electrode of a unipolar or bipolar lead with the device housing or can 60.

The sensing circuitry of the device generates atrial and ventricular electrograms from the biopotential signals sensed by the electrodes of a particular channel. An electrogram is analogous to a surface ECG and indicates the electrical activity that occurs as the heart beats. When the amplitude of an electrogram signal in an atrial or sensing channel exceeds a specified threshold, the controller or a separate comparator circuit detects an atrial or ventricular sense, respectively, which indicates depolarization occurring in the chamber. Atria and ventricular senses may also be referred to as P-waves and R-waves, respectively, in correspondence with their representations in a surface ECG. The controller uses chamber sense signals in pacing algorithms in order to trigger or inhibit pacing and to derive heart rates by measuring the time intervals between senses.

An electrogram contains components besides the depolarization electrogram signal. The heart depolarizes during systolic contraction and repolarizes during the subsequent diastolic relaxation. An electrogram therefore includes waveforms representing both depolarization (R-waves in the case of ventricular electrograms and P-waves in the case of atrial electrograms) and repolarization (T-waves in the case of ventricular electrograms). Since is only the depolarization component of the electrogram that is of interest in detecting a chamber sense, the repolarization component of the electrogram needs to be removed if the criterion for detecting a chamber sense is based solely upon the amplitude of the electrogram signal. Fortunately, depolarization and repolarization waveforms are relatively bandlimited signals, and their frequency components differ sufficiently enough that the repolarization waveforms can be filtered out. The frequency content of a depolarization waveform, such as an R-wave or a P-wave, ranges from 20 Hz to 90 Hz, while repolarization waveforms such as T-waves contain little energy above 10 Hz. Each of the filtering circuits 35 and 45 of the device in FIG. 1 therefore has a high-pass filter for removing the low-frequency repolarization components of the electrogram signal prior to further processing for detecting chamber senses. A low-pass filter is also included in each of the filtering circuits 35 and 45 for removing high-frequency noise from the electrogram signal due to extra-cardiac sources such as skeletal muscle and to prevent aliasing when the electrogram is sampled and digitized by an analog-to-digital converter.

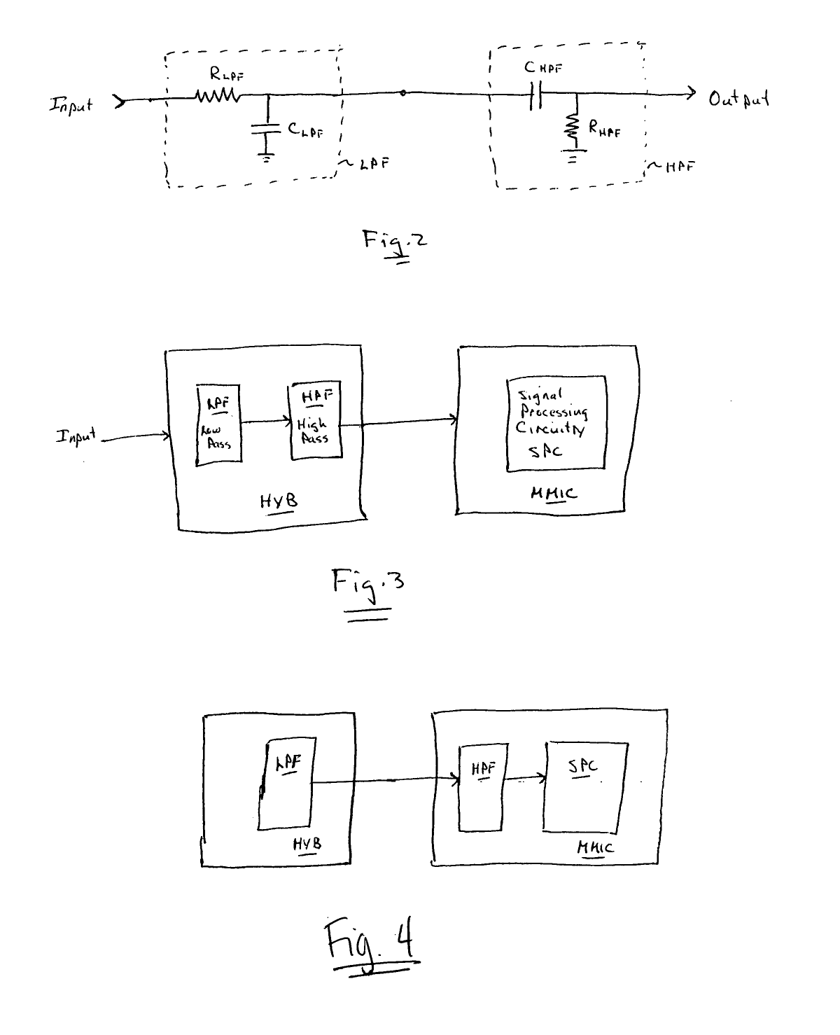

FIG. 2 shows an example of a low-pass and high-pass filtering circuit for filtering biopotential signals in an implantable cardiac device. A passive first order low-pass filter LPF is made up of a series resistor RLPF and a shunt capacitor CLPF, and a passive first order high-pass filter HPF is made up of a series capacitor CHPF and a shunt resistor RHPF. The corner frequencies of the filters are selected so as to pass a frequency range corresponding to that of an atrial or ventricular depolarization waveform. The corner frequency of each filter is determined by its RC time constant. Example values of the resistors and capacitors which result in appropriate corner frequencies are: R_{LPF}=200 Kohms, C_{LPF}=2.85 nF, R_{HPF}=1 Mohm, and C_{HPF}=14 nF. The signal processing circuitry of devices such as shown in FIG. 1 (i.e., the sense amplifiers and channel interfaces) are preferably constructed as part of a monolithic integrated circuit chip, both for reasons of efficiency and to minimize space, which is important in any implantable device. The filter circuits 35 and 45, due to their required resistance and capacitance values, are typically constructed as discrete components. FIG. 3 shows a design scheme for a sensing channel which has been used in prior devices where the signal processing circuitry SPC is incorporated into a multi-modal integrated circuit MMIC, while the filters LPF and HPF are implemented discretely on a construct referred to as a hybrid HYB. The discrete components of the high-pass and low-pass filters, in addition to taking up space, are the largest contributors to channel gain variation. It would be desirable if some of the functionality of the filter circuits could be moved to the integrated circuit chip.

FIG. 4 shows an improved design for a sensing channel in which the low-pass filter LPF is implemented on the hybrid HYB, while the high-pass filter HPF is fabricated on the integrated circuit chip MMIC. A difficulty arises with implementing this design, however, due to the required capacitance and resistance values for the high-pass filter. In the example values for the high-pass filter given above, a capacitance of 14 nF and a resistance of 1 Mohm results in an RC time constant of 14 ms. With current integrated chip fabrication processes, a 14 nF capacitor would require a chip area on the order of 45,000 m 2 which is obviously unacceptable. If the capacitance value were to be reduced to 50 pF, the resistance value which gives the same RC time constant of 14 ms is 280 Mohms. A 50 pF capacitor can be fabricated on an integrated circuit chip with acceptable dimensions, but a 280 Mohm resistor would require a physical length on the order of 20 cm which is again not acceptable. A solution to this problem is to implement the resistor as a switched capacitor circuit.

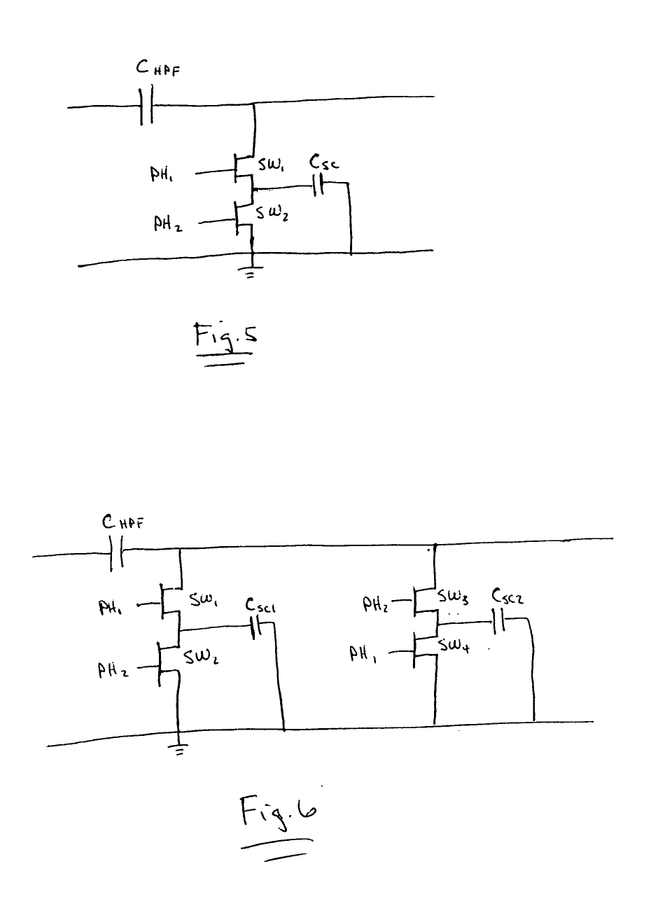

A passive high-pass filter utilizing a switched capacitor suitable for fabrication on an integrated circuit chip is illustrated in FIG. 5. The filter includes a series capacitor C_{HPF} and a shunt resistance R_{eq} formed by the switched capacitor circuit made up of a capacitor C_{sc} and switches SW₁ and SW₂. The capacitor C_{sc} is switched between the output node and ground by the switches at a selected switching frequency. The switches are driven at their gates by a two-phase clock which provides complementary but non-overlapping clock phases PH₁ and PH₂. In this circuit, the capacitance of the switched capacitor C_{sc} is selected to be 0.25 pF, which is an acceptable value for fabrication purposes. At a switching frequency F of 16 KHz, the equivalent resistance R_{eq} of the switched capacitor circuit is then R_{eq}=(C_{sc}F)⁻¹=250 Mohms. A capacitance value for the series capacitor CHPF which results in the desired RC time constant of 14 ms would then be calculated as CHPF=(14 ms/250 Mohms)=56 pF, which is an acceptable value for fabrication purposes.

An alternate implementation of a switched capacitor passive high-pass filter is illustrated in FIG. 6. This design, which may be called a balanced capacitor implementation, utilizes the same series capacitor CHPF as in FIG. 5 and two switched capacitors C_{sc1} and C_{sc2} which are each switched alternately between the output node and ground by switches SW₁ through SW₄ driven by clock phases PH₁ and PH₂. While one of the switched capacitors is switched to the output node, the other is switched to ground. The two switched capacitors thus form two equivalent resistances in parallel. If the capacitances of C_{sc1} and C_{sc2} are each made the same as that of C_{sc} in FIG. 5, halving the switching frequency to 8 KHz gives the same equivalent resistance as the circuit of FIG. 5 to result in an RC time constant of 14 ms.

Although the invention has been described in conjunction with the foregoing specific embodiments, many alternatives, variations, and modifications will be apparent to those of ordinary skill in the art. Other such alternatives, variations, and modifications are intended to fall within the scope of the following appended claims.

1. An implantable medical device, comprising:
a sensing electrode for sensing biopotential signals; and,
a high-pass filter for removing low-frequency components from the sensed biopotential signals, wherein the high-pass filter is a passive circuit having a series capacitance and a shunt resistance and further wherein the shunt resistance is formed by a switched capacitor;
circuitry for processing the biopotential signals; and,
wherein the high-pass filter and processing circuitry are fabricated on a monolithic integrated circuit chip.

2. The device of claim 1 wherein the shunt resistance is formed by a switched capacitor and two switches which switch the capacitor between an output node and ground at a selected switching frequency.

3. The device of claim 1 wherein the shunt resistance is formed by two switched capacitors and four switches which switch each of the capacitors alternately between an output node and ground at a selected switching frequency.

4. The device of claim 1 further comprising a low-pass anti-aliasing filter for removing high-frequency components from the sensed biopotential signals prior to high-pass filtering.

5. The device of claim 4 wherein the low-pass filter is a passive circuit having a series resistor and a shunt capacitor.

6. The device of claim 4 wherein the low-pass filter is located off of the integrated circuit chip.

7. The device of claim 1 wherein the sensing electrode is adapted for disposition in a patient to sense electrogram signals.

8. The device of claim 2 wherein the capacitance of the switched capacitor is approximately 0.25 pF, and the selected switching frequency is approximately 16 KHz.

9. The device of claim 8 wherein the series capacitance is approximately 56 pF.

10. The device of claim 3 wherein the capacitance of each the switched capacitors is approximately 0.25 pF, and the selected switching frequency is approximately 8 KHz.

11. A sensing circuit for an implantable medical device, comprising:
a high-pass filter for removing low-frequency components from a biopotential signal, wherein the high-pass filter is a passive circuit having a series capacitance and a shunt resistance and further wherein the shunt resistance is formed by a switched capacitor;
an amplifier for amplifying the biopotential signal after high-pass filtering; and,
wherein the high-pass filter and the amplifier are fabricated on a monolithic integrated circuit chip.

12. The circuit of claim 11 wherein the shunt resistance is formed by a switched capacitor and two switches which switch the capacitor between an output node and ground at a selected switching frequency.

13. The circuit of claim 11 wherein the shunt resistance is formed by two switched capacitors and four switches which switch each of the capacitors alternately between an output node and ground at a selected switching frequency.

14. The circuit of claim 11 further comprising a low-pass anti-aliasing filter for removing high-frequency components from the sensed biopotential signals prior to high-pass filtering.

15. The circuit of claim 14 wherein the low-pass filter is a passive circuit having a series resistor and a shunt capacitor.

16. The circuit of claim 4 wherein the low-pass filter is located off of the integrated circuit chip.

17. The circuit of claim 1 wherein the circuit is adapted for sensing electrogram signals.

18. The circuit of claim 12 wherein the capacitance of the switched capacitor is approximately 0.25 pF, and the selected switching frequency is approximately 16 KHz.

19. The circuit of claim 18 wherein the series capacitance is approximately 56 pF.

20. The circuit of claim 13 wherein the capacitance of each the switched capacitors is approximately 0.25 pF, and the selected switching frequency is approximately 8 KHz.
